(19) United States
(12) Patent Application Publication
(10) Pub. No.: US 2022/0241529 A1
(43) Pub. Date: Aug. 4, 2022

(54) GAS VALVE SYSTEM WITH MANIFOLD

(71) Applicant: OXYPOINT NV, Antwerpen (BE)

(72) Inventors: Philip HENDRICKX, Antwerpen (BE); Cedric VAN STEENKISTE, Lint (BE); Niels PINTENS, Borgerhout (BE); Wouter VAN ROOST, Hofstade (BE); Paul HERMANS, Hoboken (BE); Dirk BORGONJON, Kalmthout (BE)

(21) Appl. No.: 17/727,417

(22) Filed: Apr. 22, 2022

Related U.S. Application Data
(63) Continuation-in-part of application No. PCT/EP2020/079838, filed on Oct. 22, 2020.

(30) Foreign Application Priority Data
Oct. 22, 2019 (EP) 19204732.2
Nov. 17, 2019 (EP) 19209612.1
Nov. 18, 2019 (EP) 19209878.8
Dec. 30, 2019 (EP) 19220017.8
Dec. 30, 2019 (EP) 19220019.4
Dec. 30, 2019 (EP) 19220020.2

(51) Int. Cl. A61M 16/00; A61M 16/08

(52) U.S. Cl. CPC A61M 16/024; A61M 16/0051; A61M 16/0816; A61M 2205/3331; A61M 2016/0027; A61M 2016/003; A61M 2202/0208

(57) ABSTRACT

A gas valve system adapted for controlling the flow of a gas for a user is disclosed. The gas valve system comprises a housing, at least one sensor, one or more electronic and/or mechanical components for controlling the gas flow, and a manifold comprising one or more channels for guiding the gas between an input and an output of the gas valve system and to which the at least one sensor and one or more electronic and/or mechanical components can be coupled.

### FIELD OF THE INVENTION

The invention relates to the medical field. More particularly, the present invention relates to systems and methods for administering gas to a patient.

### BACKGROUND OF THE INVENTION

The use of gases in medical applications is widespread. One of the gasses often administered to patients for medical reasons is oxygen. In view of the fact that administering oxygen is often essential for preventing damage to tissue, avoiding life-threatening situations or saving a patient from a life-threatening situation, hospitals distribute oxygen using a pipeline network up to the bed of nearly each patient. Furthermore, oxygen therapy is also widespread for homecare patients, making use of pressurised oxygen bottles and or oxygen concentrators. In both cases, a gas valve system is used for controlling the flow, i.e., between 1 l/min and 15 l/min or higher, and the type of delivery, i.e., continuous flow or on demand flow.

A plurality of gas valve systems is available on the market, both in full mechanical as well as in combined mechanical and electronic configurations.

Current gas valve systems typically comprise a number of components, mechanical and/or electronic components whereby the gas flow in the gas valve system is led from an entrance point to an exit point by tubes passing these different mechanical and/or electronic components. The assembly of such gas valve systems requires quite some effort.

Medical gasses typically are administered to patients using a nasal cannula or a mouth mask whereby nasal cannulas often are preferred in view of patient comfort. In order to connect the nasal cannula or the mouth mask to the gas valve, the gas valve system typically comprises a hose barb component. Such hose barb components are well known to the person skilled in the art. They typically are provided on one side with a threaded portion for connected, in a substantially gas tight manner, the hose barb component to the remaining part of the gas valve system and on the other side have a particular shape referred to as Christmas-tree shape, allowing to slide the tubing onto the hose barb component in an easy manner. This easy way of connecting tubing to the gas valve system, allows avoiding the need for a clamping ring for mounting tubing to the gas valve system and hence prevents loss of time when mounting the tubing to the gas valve system.

Depending on the flow rate applied and on local practice, in some cases a moisturizer, also referred to as humidifier, can be used between the gas valve system and the tubing providing the gas to the patient, to prevent drying up of the nasal mucous membrane. In those cases, the moisturizer typically is directly mounted on the gas valve using mutually matching threaded portions. In this case, the hose barb component, used for connecting the tubing of the nasal cannula or mouth mask to the remaining part of the gas valve system, typically is not used.

It has been found that hose barb components typically get lost, when switching between medical gas administration using a moisturizer and medical gas administration without using a moisturizer. This leads to loss of time for the medical staff and to the need of additional hose barb components in order to replace the lost hose barb components.

Gas valve systems for delivery of medical gas or for providing pressurized gas or air to a patient or user typically may make use of a pressurised gas bottle, gas tank or may make use of a gas supply network as available in some hospitals. The gas pressure provided by such systems may vary and typically is too high for using it directly for delivery to patients or users. Especially in systems combining both continuous delivery and on demand delivery—but not limited thereto—operation in the correct pressure range is required in order to guarantee accurate operation. For example, on demand delivery may suffer from autotriggering at too high pressure or from insensitivity and thus bad operation at too low pressure, whereas the amount of gas delivered in a continuous mode is inaccurate when it is operated at different input pressures.

### SUMMARY OF THE INVENTION

It is an object of embodiments of the present invention to provide good gas valve systems and methods of using them.

It is an advantage of embodiments of the present invention that systems are made that allow easy manufacturing.

The present invention relates to a gas valve system adapted for controlling the flow of a gas for a user, the gas valve system comprising a housing, one or more sensors, one or more electronic and/or mechanical components for controlling the gas flow, and a manifold comprising one or more channels for guiding the gas between an input and an output of the gas valve system and to which the one or more sensors and one or more electronic and/or mechanical components can be coupled.

It is an advantage of embodiments of the present invention that the internal gas flow in the gas valve system is guided through a manifold rather than to separate tubings, allowing an easier assembling or the gas valves and more reliable system. It is an advantage of embodiment of the present invention that a rigid system is obtained.

The channels may have an average diameter of between 0.1 mm and 10 mm, for example between 1 mm and 5 mm.

One or more of the channels in the manifold may comprise one or more local channel diameter reductions, also referred to as orifice ring. It is an advantage of embodiments of the present invention that there is no need for a separate raster component which typically needs to be introduced in the gas tubing for allowing accurate operation of the gas valve system.

The local channel diameter reduction, also referred to as orifice ring, may be provided by local protrusions of the channel wall into the channel. It is an advantage of embodiments of the present invention that local channel diameter reductions can be easily introduced during the production process of the manifold.

The channel may comprise openings in the channel wall before and after the local channel diameter reduction, to which a flow sensor can be coupled for measuring the flow of the gas in a bypass channel. It is an advantage of embodiments according to the present invention that by providing local channel diameter reductions, the system can be used with a smaller sensor for measuring the flow and/or smaller electronic and/or mechanical components.

The openings in the channel wall may have an axial direction perpendicular to the overall longitudinal direction of the gas channel.

The openings in the channel wall may have an axial direction non-perpendicular to the overall longitudinal direction of the gas channel.

The manifold may be made of a single piece comprising the channels. The manifold may be made in any suitable manner, such as for example by moulding, by 3D printing, by processing a block of material, etc.

The manifold may be made of any suitable material, such as for example it may for example be made of a plastic or a metal.

The manifold may comprise alignment features for aligning one of the one or more sensors and/or one or more electronic or mechanical components with respect to the channels in the manifold. It is an advantage of embodiments of the present invention that easy positioning of the different components can be obtained during assembly of the gas valve system.

The present invention also relates to a manifold for use in a gas valve system, the manifold comprising one or more channels for guiding a gas between an input and an output of the gas valve system and to which the one or more sensors and one or more electronic and/or mechanical components can be coupled. The one or more channels in the manifold may comprise one or more local channel diameter reductions, also referred to as orifice ring.

The local channel diameter reduction may be provided by local protrusions of the channel wall into the channel.

The channel may comprise openings in the channel wall before and after the local channel diameter reduction, to which a flow sensor can be coupled for measuring the flow of the gas in a bypass channel.

The openings in the channel wall may have an axial direction perpendicular to the overall longitudinal direction of the gas channel or wherein the openings in the channel wall have an axial direction non-perpendicular to the overall longitudinal direction of the gas channel.

The present invention also relates to a flow sensor for measurement of a gas flow, the flow sensor comprising one or more channels for guiding the gas between an input and an output of the one or more channels, wherein the one or more channels comprise one or more local channel diameter reductions and wherein the channel comprises openings in the channel wall before and after the local channel diameter reduction, to which a flow sensor can be coupled for measuring the flow of the gas in a bypass channel.

The present invention also relates in another aspect to a gas valve system adapted for controlling the flow of a gas for a user, the gas valve system comprising an input portion for connecting the gas valve to an external supply, a regulating system for regulating the flow of the gas and an output portion for providing the controlled flow of the gas towards the user, the output portion having a tubular shaped ending with a threaded portion for releasably connecting a hose barb component suitable for connecting tubing to the gas valve, characterised in that the output portion furthermore has a protrusion upstream the threaded portion, and the system having a linking element being connected to the output portion of the gas valve system upstream the protrusion and being suitable for fixedly linking the hose barb component to the output portion of the gas valve system.

Embodiments of the present invention thus provide, when the hose barb component is mounted on the gas valve system a first releasable connection between the hose barb component and the gas valve via their threaded portions to provide gas flow from the gas valve through the hose barb component towards the user and a second fixed connection between the hose barb component and the gas valve system via the linking element for fixedly connecting the hose barb component to the gas valve, allowing for example to prevent loss of the hose barb component when the hose barb component is not in use, e.g., when a humidifier such as an aqua pack is connected to the gas valve.

It is an advantage of embodiments of the present invention that the hose barb is fixedly linked to the output portion so that, when the hose barb component is loosened from the threaded portion of the output portion, the hose barb component is still fixedly linked to the output portion and the hose barb component cannot be lost when it is not in use for connecting the gas valve system with tubing towards a user.

The protrusion may be ring-shaped and is extending around the output portion. It is an advantage of a protrusion that is extending around the output portion, e.g., along the circumference of the tubular shaped portion of the output portion, that the linking element cannot slip off the output portion and that the hose barb component thus has a good, fixed connection to the output portion.

The linking element may comprise an opening with a diameter smaller than an outer diameter of the protrusion and the output portion may pass through the opening at the position upstream the protrusion of the output portion.

The linking element may be a flexible element. It is an advantage of embodiments of the present invention that the linking element is a flexible element allowing an easier releasing the releasable connection of the hose barb component at its threaded portion.

The linking element may comprise a strip of solid flexible material. In some embodiments, the linking element may be made of a strip of solid flexible material.

The strip of solid flexible material may be made of any of a plastic material or a rubber material.

The linking element may comprise a chain.

The hose barb component may be a part of the gas valve system. The hose barb component may have a threaded portion for obtaining said releasable connection to the output portion via the threaded portion of the tubular shaped ending of the output portion and the hose barb component furthermore may be connected to the linking element for providing said fixed connection between the hose barb component and the output portion of the gas valve system.

The hose barb component may comprise a portion with a Christmas tree shape.

In one aspect, the present invention also relates to a gas valve system adapted for controlling the flow and/or pressure of a gas for a user, the gas valve system comprising at least one sensor for measuring the output flow of the gas supplied by the gas valve system, so as to determine the amount of gas delivered during a predetermined period. The gas may be a medical gas such as for example oxygen. In some embodiments, the gas also may be pressurized air. It is an advantage of embodiments of the present invention that the actual amount of gas delivered is registered, rather than a planned amount of gas. The latter allows to have a more accurate view on the actual therapy applied and on the actual usage of gas for a patient, a department or a building.

The gas valve system may comprise a memory for storing the amount of gas delivered during a predetermined period. It is an advantage of embodiments of the present invention that the information can be locally stored and that the gas valve can function as an independent device.

The gas valve system may comprise a user identification means and the gas valve system may be adapted for determining or storing the amount of as delivered during a predetermined period for an identified user, for a given group of users, for a hospital or care institution department or for a hospital or care institution or home care environment. It is an advantage of embodiments of the present invention that the amount of gas used per user can be recorded. The latter may assist in an improved use of gas and/or better therapy advice. It is to be noted that, in embodiments wherein vital parameters are collected, these also may be taken into account for obtaining improved gas use and/or for obtaining better therapy advice.

The gas valve system may comprise a processor for determining a degree of therapy loyalty of the user, the therapy loyalty expressing how good the user is applying a prescribed gas therapy. The gas valve system may be configured for identifying when a patient does not follow a predetermined gas therapy. The gas valve system may be configured for identifying when a patient does not follow a predetermined gas therapy by means of a visual signal, an audio signal, a haptic signal or a gas pressure signal. The visual signal may for example be a warning light or may be more detailed, such as for example the display of numerical or graphical information regarding the breathing or the gas therapy.

The gas valve system may comprise at least one channel for guiding the gas between an input and an output, the channel comprising one or more local channel diameter reductions and wherein the at least one sensor for measuring the output flow of the gas supplied by the gas valve system is adapted for measuring the flow of the gas in a bypass channel of the at least one channel with one or more local channel diameter reductions.

In one aspect, the present invention also relates to a gas valve system adapted for controlling the flow and/or pressure of a gas for a user, the gas valve system comprising

a local controller for controlling the flow and/or pressure and/or way of delivery of gas supplied to the user, and

a remotely positioned controller in communication with the local controller, the remotely positioned controller being configured for remotely obtaining information regarding the flow and/or pressure and/or the way of delivery of gas supplied to a user from the local controller and being configured for adjusting the local controller for adjusting the flow and/or pressure and/or way of delivery of the gas supplied to the user.

It is an advantage of embodiments of the present invention that the delivery of gas can be controlled and/or adapted from a distance. The latter allows for example that infection risks for medical staff can be reduced since they do not need to approach the user closely. It is an advantage that a gain in time can be obtained for medical staff, since the need for wearing protective clothes is not present anymore in view of the remote handling and the time required for applying protective clothes can be avoided. Yet another advantage is that this can enable and facilitate telemedicine as the caregiver can provide care and follow-up the given care from a distance.

In one aspect, the present invention also relates to a gas valve system for supplying a gas for a user, the gas valve system comprising at least one pressure or flow sensor and one or more electronic and/or mechanical components for controlling the gas flow, the one or more electronic and/or mechanical components for controlling the gas flow comprising at least one proportional pressure gauge, and a controller for controlling the at least one proportional pressure gauge for adapting the gas release during a pulse of an on-demand gas delivery.

The proportional pressure gauge may for example be a piezo-electric pressure gauge, a servo pressure gauge or a solenoid pressure gauge. The proportional pressure gauge typically may be configured for providing an amount of gas that is proportional with the setting of the pressure gauge.

The controller may be adapted for controlling one or more of a bolus height, a length of delivery or a triggering moment for delivering the gas.

The controller may take into account information regarding one or more vital parameters for said controlling. The controller may take into account information regarding the breathing cycle, such as for example a measured or predicted moment of inhalation.

The controller may be adapted for providing delivery of the gas prior to the actual breathing in by the user.

Particular and preferred aspects of the invention are set out in the accompanying independent and dependent claims. Features from the dependent claims may be combined with features of the independent claims and with features of other dependent claims as appropriate and not merely as explicitly set out in the claims.

These and other aspects of the invention will be apparent from and elucidated with reference to the embodiment(s) described hereinafter.

### BRIEF DESCRIPTION OF THE DRAWINGS

FIG. 1 illustrates an elevated top view of a system according to an embodiment of the present invention.

FIGS. 2 to 7 illustrate different side views, top and bottom views and cross-sectional views of the system as indicated in FIG. 1;

FIG. 8 illustrates the manifold with additional components, as can be used in an embodiment according to the present invention.

FIG. 9 illustrates a manifold with connectors according to an embodiment of the present invention.

FIG. 10 illustrates a gas valve system according to an embodiment of the present invention.

FIG. 11 illustrates a flow sensor according to an embodiment of an aspect of the present invention.

FIG. 12 illustrates another example of a gas valve system according to an embodiment of the present invention.

FIG. 13 illustrates the principle of a gas pressure reducing element, as used in embodiments of the present invention.

FIGS. 14 and 15 illustrate a connector or connector add-on having a built in gas pressure reducing element, according to embodiments of the present invention.

FIG. 16 to FIG. 20 illustrate a gas valve system comprising a linking element, according to embodiments of one aspect of the present invention.

FIG. 21 illustrates a schematic overview of a pneumatic configuration of a gas valve system according to an embodiment of one aspect of the present invention.

FIG. 22 illustrates different views of a gas valve system according to an embodiment of one aspect of the present invention.

FIG. 23 illustrates an exemplary schematic overview of an electronic scheme of a gas valve system according to an embodiment of one aspect of the present invention.

FIG. 24 illustrates a schematic overview of a gas valve system that can be controlled from a distance, according to an embodiment of one aspect of the present invention.

FIG. 25 to FIG. 32 illustrate different possibilities for adjusting the release during a pulse of an on-demand controlled gas valve system, according to embodiments of one aspect of the present invention.

The drawings are only schematic and are non-limiting. In the drawings, the size of some of the elements may be exaggerated and not drawn on scale for illustrative purposes.

Any reference signs in the claims shall not be construed as limiting the scope.

In the different drawings, the same reference signs refer to the same or analogous elements.

### DETAILED DESCRIPTION OF ILLUSTRATIVE EMBODIMENTS

The present invention will be described with respect to particular embodiments and with reference to certain drawings, but the invention is not limited thereto but only by the claims. The dimensions and the relative dimensions do not correspond to actual reductions to practice of the invention.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the below description, gas valve systems are described adapted for controlling the flow of a gas for a user, such as for example oxygen gas for oxygen gas therapy. Alternatively, the gas involved may for example be pressurized air, such as for example in sleep apnea applications. The gas valve system may be for use in hospital, for use in care institutions, for use in home care applications or may be for mixed use. The gas valve system may be for continuous flow and/or may be for on demand use. Where reference is made to on demand gas valve systems or gas valve systems operated in on demand mode, reference is made to gas valve systems wherein no continuous gas delivery is provided but wherein gas delivery is based on a trigger, e.g., breathing inhalation of the user. In some embodiments according to the present invention, the gas valve system may be as described for example in international patent application WO2012/153293, although embodiments are not limited thereto. The gas valve system may according to some embodiments be based on only mechanical components or may be based on a combination of mechanical and electronical components. Particular features such as for example the amount of gas that can be delivered (e.g., at least up to 10 liter/min., or at least up to 15 liter/min. or at least up to 25 liter/min., or even at least up to 40 liter/min.) may be as described in WO2012/153293, although embodiments are not limited thereto.

In a first aspect, the present invention relates to a gas valve system adapted for controlling the flow and/or pressure of a gas for a user. The gas may be a medical gas such as for example oxygen. In some embodiments, the gas also may be pressurized air. The gas valve system according to embodiments of the present invention may be a gas valve system for delivering continuous gas flow to a patient, for administering gas flow on demand, or for allowing both continuous gas administering or on demand gas administering. The gas valve system is not limited to a system for a particular type of gas. Depending on the properties of the gas to be delivered, the different settings of the components may be adapted. Furthermore, also the particularities of the design of the different components may be adapted. The gas valve system according to an aspect of the present invention comprises a housing. Such a housing may be made in any suitable material and may be of any suitable shape. In the housing, at least one sensor, for example two sensors, such as for example one or more sensors selected from the group of absolute pressure sensors, flow sensors, differential pressure sensors, etc. or a mixture thereof is provided. The gas valve system also comprises one or more electronic or mechanical components, such as for example valves like needle valves, for controlling the amount of gas flow that is to be administered. The gas valve system also may comprise an electronic or mechanical input means for allowing a user to provide the required input.

According to embodiments of the present invention, the gas valve system also comprises a manifold comprising one or more channels for guiding the gas between an input and an output of the gas valve system and to which the one or more sensors and one or more electronic and/or mechanical components can be coupled. The channels in the manifold may be fixed channels formed in the material of the manifold. The channels may have a diameter between 0.1 mm and 10 mm, e.g., between 0.5 mm and 5 mm. The cross-sectional shape of the channels may depend on the manufacturing technique of the manifold. The channels may have a square cross section, a rectangular cross section, a circular cross-section etc.

The manifold may be made of a single piece comprising the channels, but also may comprise of a plurality of pieces. In some embodiments, two major parts of the manifold may be present, whereby the two parts fit together and can be mounted to form a single piece. Alternatively, the manifold also could be made of a single piece. The manifold may be made in a plurality of manners, such as for example using moulding, using casting, using processing of a block of material, using 3D printing, and alike. The manifold may be made of any suitable material such as for example made of plastic, of metal, etc.

The manifold also may comprise alignment features for aligning one of the one or more sensors and/or one or more electronic or mechanical components with respect to the channels in the manifold.

The gas valve system also may comprise an output means for displaying information regarding the gas delivery. The displaying means may be a display for graphically or numerically displaying information. The displaying means may be a visual indication, such as for example a LED ring.

In some embodiments, the gas valve system may comprise one or more channels in the manifold having one or more local channel diameter reductions, e.g., for obtaining flow reductions or measuring a flow. Such a local channel diameter reduction may overcome the need for using a honeycomb material in the channel, as typically is required in conventional systems for creating a differential pressure measurement system. The local channel diameter reduction may be provided by local protrusions of the channel wall into the channel. The channel may comprise openings in the channel wall before and after the local channel diameter reduction, to which a flow sensor can be coupled for measuring the flow of the gas in a bypass channel. The opening in the channel wall may have an axial direction perpendicular to the overall longitudinal direction of the gas channel or they may have an axial direction non-perpendicular to the overall longitudinal direction of the gas channel. The angle with respect to the overall longitudinal direction of the gas channel may be such that the amount of disturbance of the gas flow in the main channel is limited.

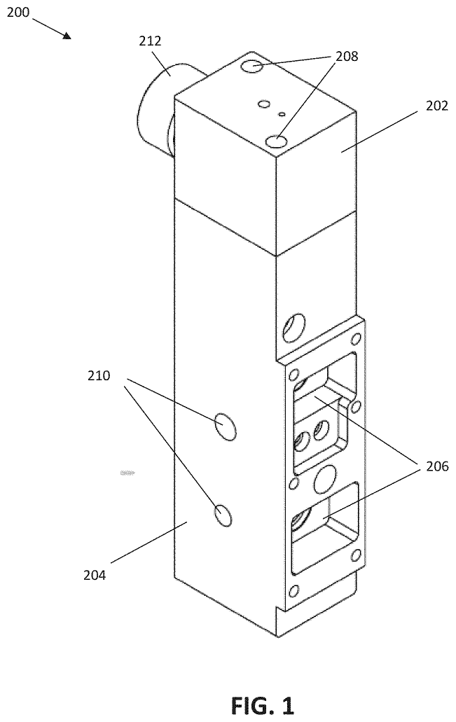

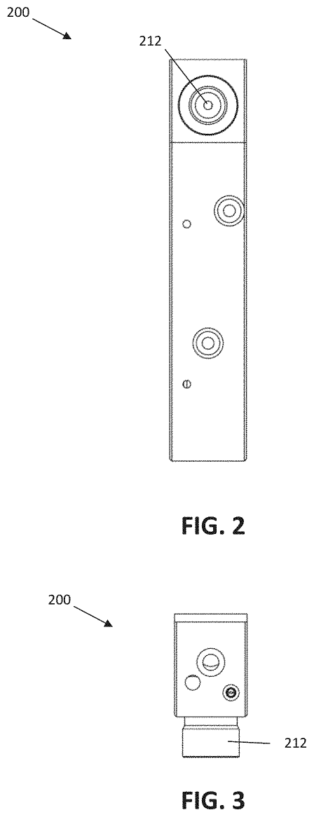

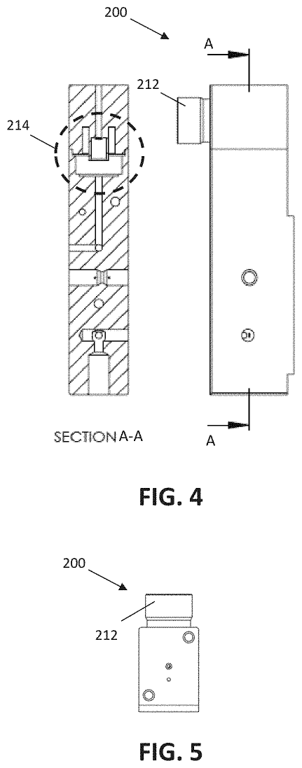

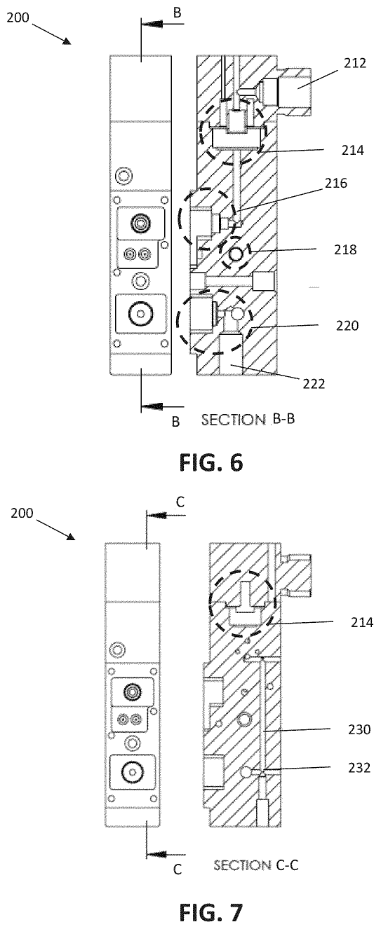

By way of illustration, embodiments of the present invention not being limited thereto, a gas valve system with manifold is described with reference to drawings FIG. 1 to FIG. 10. FIG. 1 shows an elevated view of the manifold 200. It can be seen that in the present example, the manifold 200 is built from two parts 202, 204, which allows for an easy assembly of the pressure reduction component that is present in the manifold and that will be discussed later. In FIG. 1, furthermore the positions 206 where the different sensors will be positioned can be seen. It is an advantage of embodiments of the present invention that the predefined upstanding edges and shaped features allow for an accurate positioning of the different sensors. Furthermore, mounting holes 208, 210 are shown that can be used for mounting different components to each other and/or for positioning the manifold with respect to the housing. FIG. 1 also illustrates a manifold input part 212 that is adapted for introducing the gas into the manifold. The manifold input part 212, may comprise in one example a threaded part for connecting a standard input connector to it. Such a standard input connector may be a DIN input connector, although embodiments are not limited thereto, and FIG. 2 illustrates a back view of the manifold 200. The manifold input part 212 can be easily seen. FIG. 3 illustrates a bottom view of the manifold 200. Again, the manifold input part 212 can be easily seen. FIG. 4 illustrates a side view of the manifold 200 as well as a cross-sectional view along line A-A. In the cross-sectional view, part of the pressure reduction component 214 is shown, where the pressure of the incoming gas is reduced, so as to obtain a suitable pressure for use in gas delivery. The pressure reduction may for example be towards a predetermined pressure, such as for example to a pressure in a range between 1 bar and 2 bar, such as for example 1.5 bar or for example 2 bar. FIG. 5 illustrates a top view of the manifold. wherein again the manifold input part 212 can be seen. FIG. 6 illustrates a front view of the manifold with a cross-section line B-B for which the cross-sectional view along line B-B is also shown. FIG. 7 illustrates the same front view of the manifold but with cross-section line C-C for which the cross-sectional view along line C-C is also shown. Based on these drawings, the typical flow of the gas can be described.

In FIG. 6, the manifold input part 212 can be seen along which the gas enters the manifold. As described above, this component typically may comprise a threaded portion. From the manifold input part 212, the gas is led via one or more channels towards a pressure reduction component 214 for reducing the pressure so as to obtain a pressure that is suitable for use in gas delivery. From the pressure reduction component 214, the gas is again led via one or more channels towards an absolute pressure sensor 216. This absolute pressure sensor 216 may allow for evaluating the absolute pressure for the gas. The gas is then further led through channels towards a flow sensor 218 for checking the flow that is provided and controlled by a piezo-electrical valve. The gas is then further directed to a differential pressure sensor 220. The gas is thereafter led to a manifold output part 222.

The cross-sectional view C-C of FIG. 7 allows to further illustrate the channel used as a safety channel 230 so that in case of non-functioning of one of the components at least a safety gas flow can be obtained. The flow thereof will be controlled by a needle valve 232.

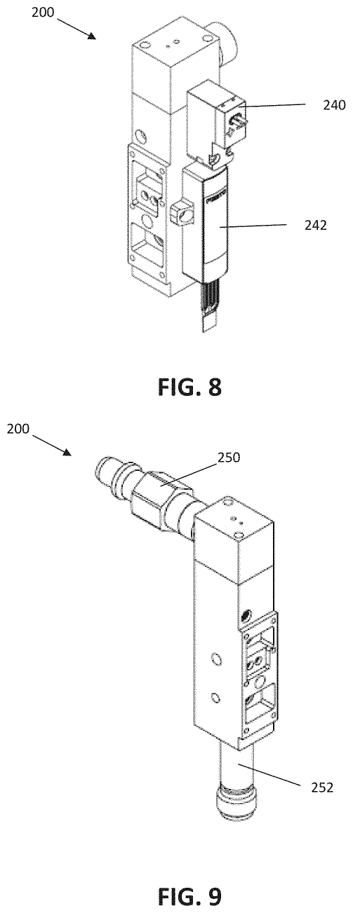

In FIG. 8, the pressure valves of which one 242 is used in the conventional gas stream for controlling the gas flow, which in the present example is a piezo-electric valve, and of which one 240 is used in the safety gas stream for controlling the gas flow, which in the present example is a latching valve, are shown, mounted on the manifold. The piezo-electric valve is provided with an electrical connector.

FIG. 9 illustrates an additional connector 250 which is mounted to manifold input part 212 and an additional connector 252 which is mounted to the manifold output part 222. The type of connectors used may depend on the type of gas connection requirements that are valid in the country of use of the gas valve. These connectors may allow for obtaining a country specific, conventional connection.

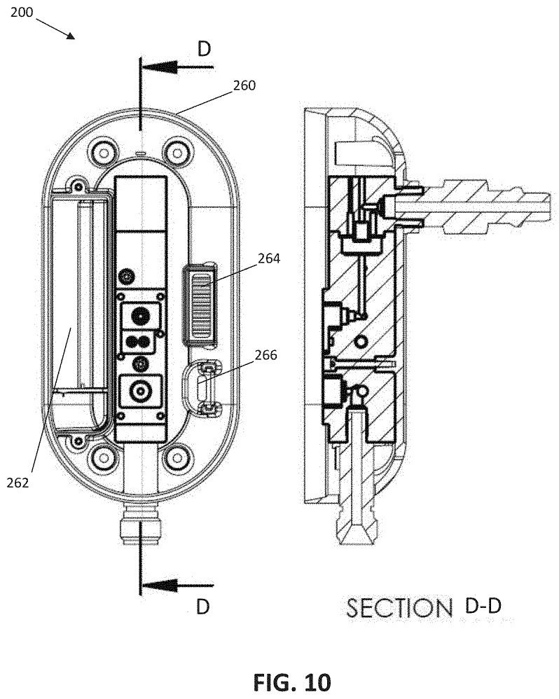

FIG. 10 illustrates how the manifold 200 is positioned in the housing 260 of the gas valve system 100. The manifold is positioned aside a space 262 where typically a battery is positioned. Furthermore, a speaker 264 and an usb connection 266 also is visible.

Further, by way of illustration, embodiments of the present invention not being limited thereto, an example of a specific flow sensor that can be used is now described. It is to be noted that the flow sensor can also be used in other gas valve systems and is therefore not limited to gas valve systems having a manifold according to embodiments according to the present invention.

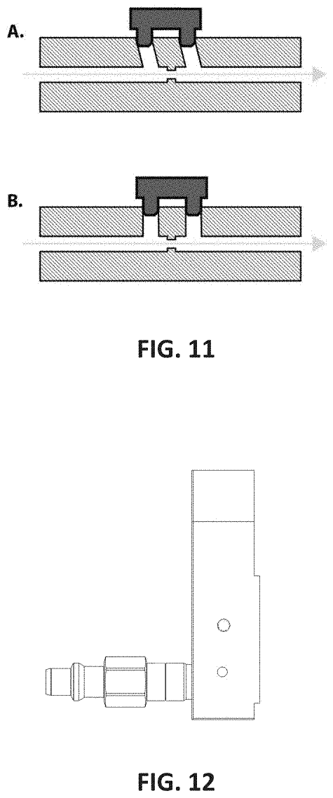

In one aspect, the present invention thus also relates to a flow sensor based on a channel wherein one or more local reductions one or more local channel diameter reductions, e.g., for obtaining flow reductions or measuring a flow. Such a local channel diameter reduction may overcome the need for using a honeycomb material in the channel, as typically is required in conventional systems. The local channel diameter reduction may be provided by local protrusions of the channel wall into the channel. The channel may comprise openings in the channel wall before and after the local channel diameter reduction, to which a flow sensor can be coupled for measuring the flow of the gas in a bypass channel. The opening in the channel wall may have an axial direction perpendicular to the overall longitudinal direction of the gas channel or they may have an axial direction non-perpendicular to the overall longitudinal direction of the gas channel. The angle with respect to the overall longitudinal direction of the gas channel may be such that the amount of disturbance of the gas flow in the main channel is limited. By way of illustration, embodiments of the present invention not being limited thereto, two examples A and B of such a configuration is shown in FIG. 11.

In one embodiment, a flow sensor for measurement of a gas flow is described. The flow sensor comprises one or more channels for guiding the gas between an input and an output of the one or more channels. The one or more channels comprise one or more local channel diameter reductions and the channel comprises openings in the channel wall before and after the local channel diameter reduction, to which a flow sensor can be coupled for measuring the flow of the gas in a bypass channel. In a particular example, the orifice ring or channel diameter reduction reduces from a diameter of 4.7 mm to 3.5 mm and enlarges again to 4.7 mm.

In one aspect, the present invention also relates to a manifold as described in the first aspect, Similar features as described for the first aspect may be present, leading to the same advantages.

By way of illustration, embodiments of the present invention not being limited thereto, a further example of a gas valve system is illustrated in FIG. 12. The gas valve system is similar to the gas valve systems as described above, but the manifold input part is positioned at a relative different position compared to the examples already illustrated. In the present example, the manifold input part is positioned more at a bottom side of the gas valve system. This illustrates that the exact position where the manifold input part is positioned relative to the housing is not limited. The inner components of the manifold may be rearranged with respect to the housing or may be as illustrated in the examples above whereby an additional connection tube or channel between the manifold input part and for example a pressure reduction component, or if the pressure reduction component is positioned outside the gas valve system, a first sensor of the gas valve system.

In some embodiments, the manifold input part may be shiftable with respect to the housing, so that the relative position can be adjusted depending on the local environment and/or application.

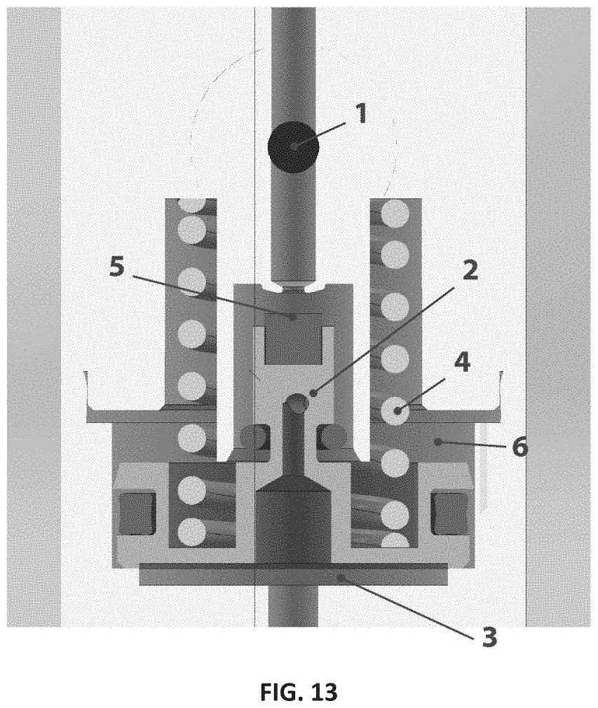

Also, by way of illustration, embodiments not being limited thereto, an example of a pressure reduction component is described in more detail, with reference to FIG. 13. The gas pressure reduction component has the function of bringing the gas pressure to a pressure that can be used for gas therapy. The working principle of the pressure reduction component may be as followed. The gas inlet 1 allows the gas to enter at its typical input pressure, which may be around 5 bar or around 6 bar depending on local conditions. The gas flows to the actuator via channel 2 being a connection in the actuator to the space 3 in the actuator. In this room the pressure builds up till a predetermined pressure sufficiently high for use in gas valve therapy, such as for example 2 bar. When the predetermined pressure is reached, a calibrated spring 4 is pressed, such that the gas flow is limited by a closing element 5, such as for example a rubber stopping element. The calibrated spring allows for the relative movement of the closing element 5 with respect to the gas inlet 1. This process occurs fast and repetitively. In this way, the pressure in the channels can be controlled and the pressure that is delivered is determined by the calibrated spring, i.e., by the power required for pressing the calibrated spring. The space 6 is in contact with the environment. The appropriate gas input pressure and output pressure can be obtained based on selection of the proper spring and proper dimensions of the system.

It is to be noted that the present invention also relates to another aspect. The present invention also relates to a gas valve system for delivering gas, which may be a medical gas or which may be for example air, to a patient or user. The system may be specifically designed for providing medical gas therapy, e.g., oxygen therapy, to a patient or may be specifically designed for providing gas or air pulses in the framework of therapy for example other purposes, such as for example in the framework of sleep apnea. Embodiments of the present invention described below may be based on a gas valve system as described in other aspects of the present invention, i.e., for example making use of a manifold, but embodiments of the present invention are not limited thereto and may for example be based on a gas valve system as described in international patent application WO2012/153293. The gas valve system may be for continuous flow and/or may be for on demand use. The gas valve system may be based on only mechanical components or may be based on a combination of mechanical and electronical components or even only on electronical components.

Gas valve systems according to embodiments of the present aspect of the present invention may be for use in hospital, for use at home or may be for mixed use. According to embodiments of the present invention, the gas valve system comprises an input portion for connecting the gas valve to an external supply. Such an external supply may for example be a pressurised gas bottle, gas tank or may be a gas supply network as available in some hospitals. It is to be noted that, especially in cases where a pressurised gas bottle or pressurised gas tank is to be used—although not limited thereto, the gas pressure with which the gas is provided by the pressurised gas bottle or pressurised gas tank, is too high for use for delivery to a patient or user. Whereas external components are known to be applied to a pressurised gas bottle or pressurised gas tank for reducing the gas pressure delivered, there is always a risk that such external components are not readily available, especially in some countries where medical gas delivery equipment is rare or less widely spread. Furthermore, even if readily available, there is always a risk that such external components are, for example by way of mistake, not introduced. The latter may result in the gas valve system to be used being destroyed, e.g., at first use, due to an overpressure. Furthermore, there is also a risk for the patient and/or user that a medical gas would be provided at a too high pressure for the patient or user.

In embodiments of the present aspect of the present invention, this problem is solved by providing an integrated gas pressure control component and/or integrated gas pressure reduction component, integrated in the gas pressure system. Where embodiments of the present invention refer to an integrated gas pressure control component or an integrated gas pressure reduction component, reference is made to a pressure control or pressure reduction component that is connected to the remainder part of the gas valve system or that is permanently connected to the remainder part of the gas valve system. In some advantageous embodiments, the pressure control component or pressure reduction component is permanently connected to the remainder part of the gas valve system, preventing the possibility that the gas pressure control or gas pressure reduction is forgotten and thus avoiding the risk of damage to the gas valve system and/or injuring of the patient or user.

According to embodiments of the present invention, the gas valve system comprises a gas pressure controlling and/or gas pressure reducing component allowing reducing the gas pressure from between 250 bar and 3 bar, e.g., between 200 bar and 3 bar, e.g., between 150 bar and 3 bar, e.g., between 50 bar and 3 bar, e.g., between 10 bar and 3 bar, e.g., from between 6 bar and 3 bar, e.g., from about 5 bar or 6 bar, to a predetermined gas pressure for use in gas therapy or other gas delivery applications. The system may be adapted for using a gas pressure reduction component or a gas pressure control component as the one described above. In some embodiments, the gas pressure control component may be a component that can be adapted to receive gas at a pressure from one of a selected number of predetermined values or from within a pressure range selected from a selected number of pressure ranges. In other embodiments, a gas pressure reduction component is used, which allows gas pressure reduction from a predetermined gas pressure or pressure range to a predetermined value at which the remainder part of the gas valve system can operate or to a predetermined value at which the gas/air should be delivered to the patient/user.

In some embodiments, the gas pressure control or reduction component may be settable to a predetermined input gas pressure or gas pressure range using a control element, such as for example a knob, a setting element or alike. In some embodiments, the gas pressure control or reduction component may for example be settable by controlling a spring or another element.

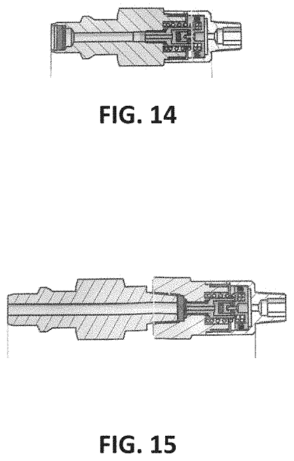

It is an advantage that a more accurate predetermined level of gas pressure is obtained at which gas delivery such as gas therapy, can be performed. It is to be noted that a more accurate calibration of the gas valve system can be obtained, since the pressure at which the gas valve system or the remainder part of the gas valve system is to be operated can more accurately be obtained or selected. It furthermore is to be noted that also further gas pressure reducing elements may be present, used in series. By way of illustration, embodiments of the present invention not being limited thereto, a gas pressure reduction component may be as described with reference to FIG. 14 and FIG. 15, as also described further below. Such pressure reduction component may be fully integrated in the gas valve system and thus may permanently fixedly be connected thereto, alternatively to a component that is mountable on the gas valve system.

It is to be noted that whereas in the above the gas pressure reducing element or component and/or the gas pressure controlling element or component has been described as an element or component providing gas pressure reduction in a single step, embodiments of the present invention also encompass exemplary gas valve systems whereby the function of gas pressure control or gas pressure reduction is performed in a two or more steps reduction, e.g., using two or more subsequent elements or components.

It is an advantage of embodiments of the present invention that an appropriate pressure reduction can be obtained, e.g., between predetermined values, so that both continuous delivery and on demand delivery can be performed in an improved way. Continuous delivery can e.g., be improved in that a more accurate delivery is performed, since the calibration is more accurate for the gas pressure that is obtained after the gas reduction is performed. With respect to on demand delivery, a more accurate delivery is obtained when a good gas pressure reduction is performed, since auto triggering of the on demand function is avoided which would occur when the pressure is too high, and a decreased sensitivity of the on the demand function is avoided, which could occur at a pressure that is too low. The gas pressure reduction and/or control may thus be advantageously used in systems combining both possibilities of continuous delivery and on demand delivery of gas. The latter may additionally be advantageous when gas containers are used that for which the pressure tends to vary when the container is used for a longer time and the amount of gas present in the container thus varies.

Whereas the gas pressure reduction element may be part of the gas valve system, it also may in one aspect be introduced in a connector element. In one aspect, the present invention thus also relates to a connector element for coupling a gas valve system to a pressurized gas bottle or a gas distribution network system, the connector element comprising a pressure reducing element. The latter is illustrated in FIGS. 14 and 15, whereby the connector can be a single connector or an additional connector that can be mounted, e.g., clicked or screwed, on an existing connector. It is an advantage of embodiments of the present invention that a pressure reducing component can be embedded in a connector. The latter allows for a compact and user friendly manner for introducing a gas pressure reducing component. It is an advantage of embodiments of the present invention that in this way an easy pressure reduction can be obtained, e.g., a double step pressure reduction can be obtained, one being provided by the conventional gas reduction element typically mounted on a gas bottle or a distribution network, and one provided by the gas pressure reducing element in the connector element. Furthermore, it can be easily seen that different connectors with gas pressure reducing elements can be coupled in series.

In another aspect, the present invention relates to a gas valve system adapted for controlling the flow of a gas for a user. The gas valve system may be for use in hospital, for use at home or may be for mixed use. The gas valve system may be for continuous flow and/or may be for on demand use. It may be a gas valve system as described for example in international patent application WO2012/153293, although embodiments are not limited thereto. The gas valve system may be based on only mechanical components or may be based on a combination of mechanical and electronical components. According to embodiments of the present invention, the gas valve system comprises an input portion for connecting the gas valve to an external supply. Such an external supply may for example be a pressurised gas bottle or may be a gas supply network as typically available in hospitals. The input portion may be as known from prior art and is therefore not further detailed here. The system also comprises a regulating system for regulating the flow of the gas. As indicated above, the present invention is not limited to the particular regulating system used. Such a system may for example be only mechanical or may comprise a combination of mechanical and electronic components. One possible example of a regulating system is described in WO2012/153293, although embodiments are not limited thereto. According to embodiments of the present invention, the gas valve system also comprises an output portion for providing the controlled flow of the gas towards the user. The gas may for example be administered to the user making use of a nasal cannula or a mouth mask. According to embodiments of the present invention, the output portion has a tubular shaped ending with a threaded portion for releasably connecting a hose barb component suitable for connecting tubing to the gas valve. The output portion furthermore has a protrusion upstream the threaded portion. The system further has a linking element being connected to the output portion of the gas valve system upstream the protrusion and being suitable for fixedly linking the hose barb component to the output portion of the gas valve system. The protrusion may be ring-shaped and extending around the output portion. The system thus typically may be configured such that the linking element is withheld from being removed from the remaining part of the gas valve system by the protrusion, e.g., withheld from being removed when no large force is applied to the linking element. A force is considered large if it is substantially higher than for example the gravitational force present when a hose barb component is connected to the linking element that is not attached to the gas valve system in another manner than through the linking element. Embodiments of the present invention provide, when the hose barb component is mounted on the gas valve system a first releasable connection between the hose barb component and the gas valve via their threaded portions to provide gas flow from the gas valve through the hose barb component towards the user and a second fixed connection between the hose barb component and the gas valve system via the linking element for fixedly connecting the hose barb component to the gas valve.

In some embodiments, the linking element may for example comprise an opening with a diameter smaller than an outer diameter of the protrusion and the output portion passes through the opening at the position upstream the protrusion of the output portion.

The linking element may be a flexible element. In some embodiments, the linking element comprises a strip of solid flexible material, for example strip of plastic material or a rubber material. In some embodiments, part of the linking element also may comprise a chain.

In some embodiments, the hose barb component is part of the gas valve system. The hose barb component typically may comprise a threaded portion for obtaining the releasable connection to the output portion via the threaded portion of the tubular shaped ending of the output portion. In these embodiments, the hose barb component also is connected to the linking element for providing said fixed link between the hose barb component and the output portion of the gas valve system. Such a connection may be performed in a variety of ways.

The hose barb component, whether it is part of the gas valve system or whether it is connectable to the gas valve system through the linking element, may comprise a portion with a Christmas tree shape, as well known by the person skilled in the art. More generally, the hose barb component may have at one side a shape suitable for easily connecting tubing such as tubing of a nasal cannula or a mouth mask.

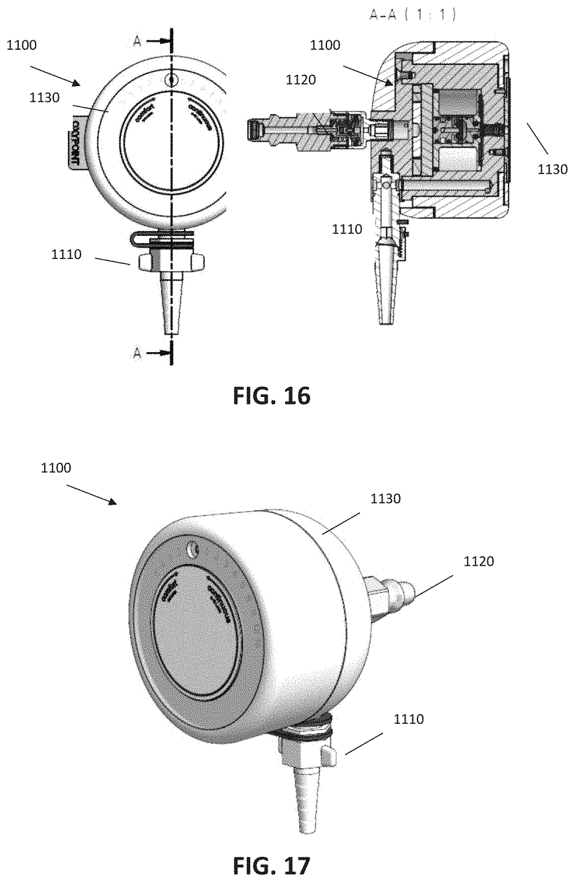

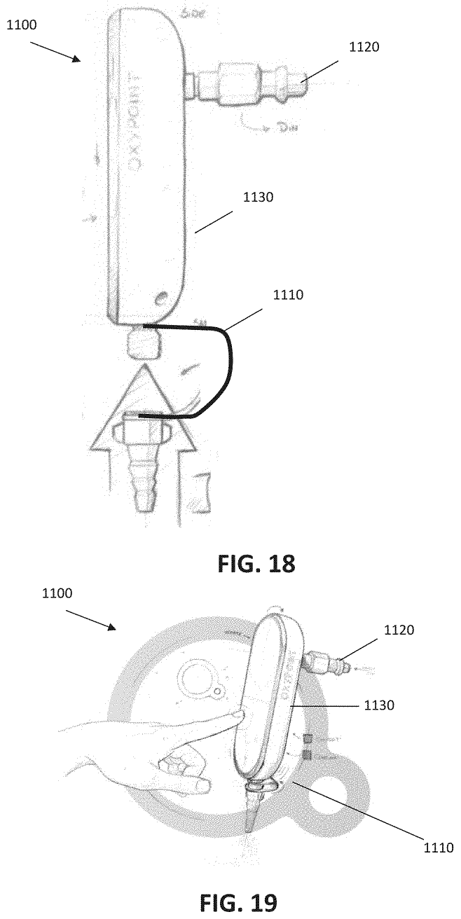

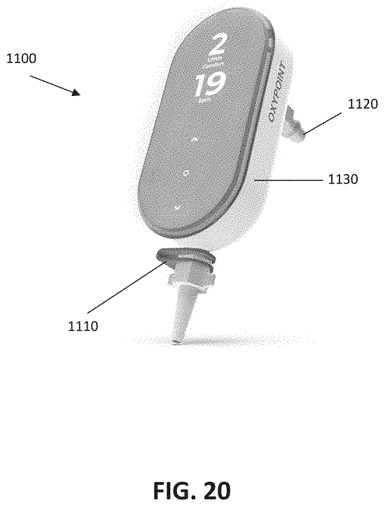

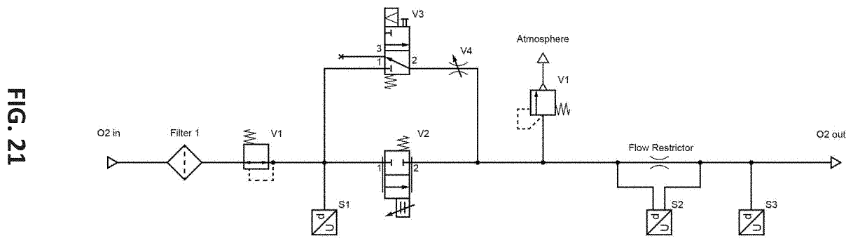

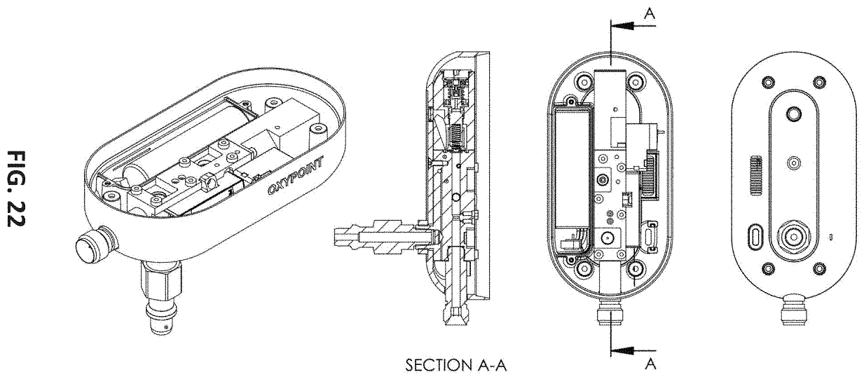

By way of illustration, embodiments of the present invention not being limited thereto, an example of an embodiment illustrating a gas valve system with a linking element is shown in FIG. 16. The gas valve system 1100 is shown with linking element 1110, connecting the hose barb component 1120 fixedly to the remaining part of the gas valve system. The remaining gas valve system also shows the input portion 1120 and the gas regulating system 1130. The gas valve system is show in front view (left hand side) and in cross-sectional view (right hand side). The detailed features of the internal part of the remaining part of the gas valve system are not limiting for the present invention, as also indicated above. Furthermore, the explicit configuration of the linking element, such as the length, the way it is connected or connectable to the hose barb component, etc. is also not limiting. Similarly, also in FIG. 17 a gas valve system is shown, in the present case with a hose barb component connected to the remaining part of the gas valve. Furthermore, also in FIG. 18 a gas valve system with a linking element is shown. The hose barb component is not being releasably connected through the threaded portions but only is fixedly connected through the linking element 1110. In this example, the input portion 1120 and the gas regulating system 1130 is also shown. FIG. 19 illustrates a similar gas valve system whereby the hose barb component is releasably connected to the remaining part of the gas valve system. FIG. 20 also shows a gas valve system according to an embodiment of the present invention.

In one aspect, the present invention also relates to a linking element adapted for use in a gas valve system adapted for controlling the flow of a gas for a user, a gas valve system as described in the above aspect.

In one aspect, the present invention also relates to a gas valve system for providing a gas to a user, that is particularly adapted for monitoring the actual output that is provided to the user. Whereas in a plurality of known systems, the amount of gas that is programmed for being delivered to the user is known, the amount that is actually delivered is not always known. According to embodiments of the present invention, a gas valve system is described that is adapted for controlling the flow and/or the pressure of a gas for a user, whereby the gas valve system comprises at least one sensor for measuring the output flow of the gas supplied by the gas valve system, so as to determine the amount of gas delivered during a predetermined period. In this way, the actual amount of gas that is delivered is registered, rather than the planned amount of the gas. The gas may be a medical gas, such as for example oxygen, or it may be for example pressurized air.

One of the major advantages of embodiments of the present invention is that a more accurate view on the actual therapy applied with the actual usage is obtained. The latter can be for a specific patient, for a department of for a building.

In some embodiments, the gas valve system comprises a memory for storing the amount of gas delivered during a predetermined period. Such a memory may for example store the amount of gas delivered, together with an identification of a patient, a department or a building. The system may for example be equipped with a patient identification system for identifying a patient so that the amount of delivered gas can indeed be coupled to a particular patient. The latter provides possibilities for monitoring and e.g., charging the gas used to a particular patient, which is advantageous especially in environments where multiple users can make use of the gas valve system. The patient identification can for example comprise an RF identification means, an NFC identification means, based on manual identification, a patient code identification means such as a bar code or QR code scanning means, identification based on Bluetooth, etc.

Alternatively, or in addition thereto, the actual use in a certain department or in a building can also be monitored more exactly.

It is an advantage of embodiments of the present invention to register the flow output of the gas over time. Embodiments may be adapted for monitoring the output and the time of delivery so that it becomes possible to obtain an amount of gas that is delivered. In some embodiments, the position of the gas valve may be used to link the output flow information to a patient. The position of the gas valve may for example be fixed, such as for example for fixed oxygen outlets in hospitals, may for example be determined by a positioning means such as for example a gps or a network-based localisation system such as for example Wi-Fi based, Bluetooth based, etc., or may in another example be provided by the user.

Embodiments of the present invention therefore render it possible to determine at patient level which amount of gas is delivered to a patient, e.g., in a hospital, in an institution for care or during use at home of the gas valve.

In some embodiments, the system can also be programmed for checking a therapy loyalty of a patient. Whereas a certain gas therapy may be subscribed by a doctor, it is not always easy to check whether the patient actually follows the prescribed gas therapy. Such therapy may be followed in a private environment, a hospital, a care institution, etc. According to some embodiments, the system thus may comprise a processor for comparing a planned therapy with the actual medical therapy followed by the patient. In some embodiments, the system may be equipped with a feedback system for providing to the patient or his medical staff feedback regarding to the compliance of the actual gas therapy followed with the planned therapy.

Whereas in the past it was difficult, if not impossible, for a medical doctor, medical staff, caring staff or other people to check whether the therapy was followed correctly, systems according to embodiments of the present invention allow for checking therapy loyalty. The system can for example allow to check whether the correct dose was used, whether breathing was performed correctly, whether the oxygen mask was worn correctly, etc. The system may even be programmed for alerting the user if one of these aspects of the therapy was missed (e.g., too much breathing through the mouth instead of through the nose). So that this can be corrected in the future.

In some embodiments, the system is configured for being used with a nose cannula and is adapted for determining whether the nose cannula is used appropriately. When a user breaths through his mouth or is even not using the nose cannula, the system may alert the user. Such an alert signal may be a visual signal, a sound signal, a haptic signal, but in some embodiments may also be a predetermined pressure signal. The system may for example be adapted for providing a series of pressure pulses through the nose cannula to warn a patient when he is breathing through the mouth, so that the user is reminded that breathing through the nose is required for the gas therapy.

In some embodiments, the system may be adapted for, when it has determined periods wherein the therapy was not followed, checking the effect of not following the therapy on e.g., the breathing parameters and/or other vital parameters. The latter may for example be used as a cross-check for evaluating the effectiveness of the therapy. It thus may provide a reference for the gas therapy, providing details of the breathing parameters and/or other vital parameters when no therapy is applied.

An example of a system wherein the actual output flow is measured may be found in a system according to the first aspect.

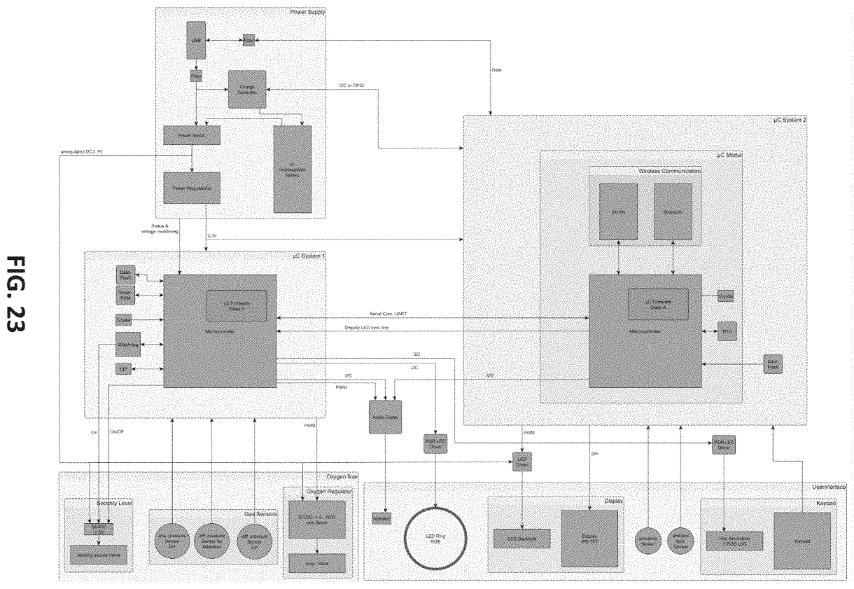

By way of illustration, embodiments of the present invention not being limited thereto, a pneumatic schedule of an exemplary gas valve system according to an exemplary embodiment is shown in FIG. 21. The measurement system for measuring the actual output flow is shown in component S2.

In one aspect, the present invention also relates to a system for detecting and preventing snoring or sleep apnea. The system may use a gas valve as described above whereby a signal, e.g., pressure signal, is applied at the moment snoring or a sleep apnea event is detected or predicted. It may be advantageous that a pressure signal is synchronised with a snoring or sleep apnea event only and is not continuously applied during breathing which enables a better adapted sleep care.

In one aspect, the present invention also relates to a gas valve system adapted for controlling the flow and/or pressure of a gas for a user from a distance. The gas may be a medical gas. In some embodiments, the gas may be pressurized air. The latter may for example be useful in sleep apnea applications. The latter may be particularly relevant in cases where a patient is infectious and the patient is to be treated in quarantine or isolation, or for example where a patient is located at a different location than the medical staff, for example when applying gas therapy such as oxygen therapy in a home or residential care situation. According to embodiments of the present invention, the gas valve system comprises a local controller for controlling the flow and/or pressure and/or way of delivery of gas supplied to the user, and a remotely positioned controller in communication with the local controller, the remotely positioned controller being configured for remotely obtaining information regarding the flow and/or the way of delivery of gas supplied to a user from the local controller and being configured for adjusting the local controller for adjusting the flow and/or way of delivery of the gas supplied to the user. Communication between the remotely positioned controller and the local controller may be performed based on predetermined protocols. The communication may be performed using commonly known communication techniques such as using a wired or wireless protocol, over the internet, via Bluetooth, or via any other means. The communication technique used may be selected as function of the particular situation, which may for example be different when home therapy is used or when communication with patients in quarantine or isolation is envisaged. It is an advantage of embodiments of the present invention that the delivery of gas can be controlled and/or adapted from a distance. The latter allows for example that infection risks for medical staff can be reduced since they do not need to approach the user closely. It is an advantage that a gain in time can be obtained for medical staff, since the need for wearing protective clothes is not present anymore in view of the remote handling and the time required for applying protective clothes can be avoided.

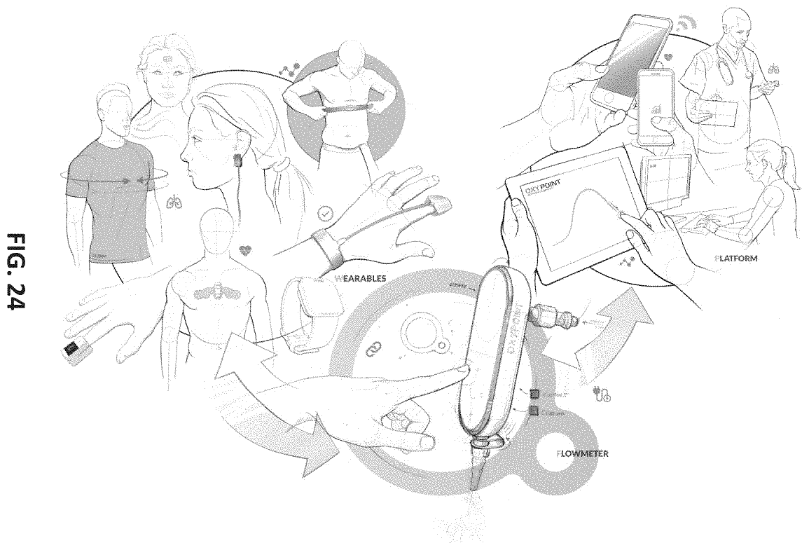

The local controller can be positioned at any suitable position in the gas valve system. By way of example, a gas valve system as shown in FIG. 22, which is comparable to the gas valve system as shown in FIG. 10 with slight mechanical and configurational adaptations, can be used. By way of illustration, embodiments of the present invention not being limited thereto, an example of a possible electronic scheme with a local controller and with components for guaranteeing communication with an external, remote controller, is shown in FIG. 23.

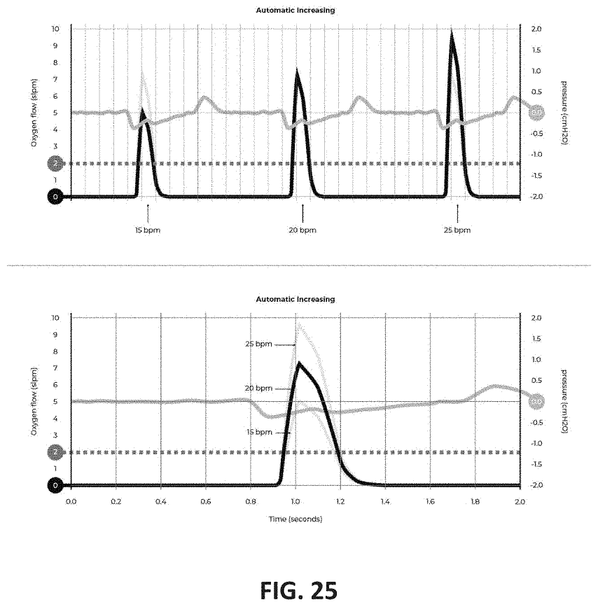

Further by way of illustration, FIG. 24 illustrates a possible configuration wherein a remotely controlled gas valve is illustrated. In the example, the gas valve is controlled using a remotely positioned controller embedded in a processor of a tablet.

In yet another aspect, the present invention relates to a gas valve system for supplying a gas, the gas valve system comprises at least one pressure or flow sensor and one or more electronic and/or mechanical components for controlling the gas flow. According to embodiments of the present invention, the one or more electronic and/or mechanical components for controlling the gas flow comprise at least one proportional pressure gauge and a controller for controlling the at least one proportional pressure gauge for adapting the gas release during a pulse of an on-demand gas delivery. The system thus overcomes the typical limited operation of existing on-demand gas delivery systems.

On-demand gas delivery systems typically are configured to operate in a particular manner. For example, on-demand systems are known that operate with a constant volume per minute. In such systems, the bolus height during the pulse is reduced when the number of breaths per minute is increasing, while the bolus height during the pulse is increased when the number of breaths per minute is decreasing. Whereas the bolus height is adapted as a function of the number of breaths per minute so that the gas valve system is providing a constant volume per minute, for a given number of breaths per minute, the bolus height during the different pulses is constant.

There also exist other on-demand valves which are based on a constant bolus height. In these systems, the valve opens for a period of time for providing a certain amount of gas. The period of time during which the valve opens is equal for subsequent pulses in a certain on-demand setting. The period of time is only varied if a higher flow setting is selected for the on-demand valve. For these systems, an increased breathing rate will result in an increased number of equal doses, so that on average per minute more gas is delivered. Similarly, when the breathing rate is decreased, the number of equal doses also will be decreased.

According to embodiments of the present invention, the gas valve system is adapted for adjusting the gas pulse during a pulse in on-demand operation a function of the breathing frequency. In this way, with a single system, the gas pulse release can be selected for a pulse in on-demand operation and can for example be fit to predetermined therapies or can be selected depending on the condition or activity of the patient using the gas therapy. The gas pulse release can be adapted in a plurality of ways: the volume can be kept constant, can be increased or decreased, the trigger speed for delivery can be adapted, the period of release of gas can be adapted, etc. such that full control of the gas release can be used.

In one example, such a system may for example be adapted for increasing the bolus when the patient is breathing faster. The latter may for example be useful during revalidation exercises. If the patient, due to the exercises, requires more oxygen and therefore starts breathing faster, the system can provide an increased bolus. Such a system can for example avoid that during exercises, a trainer needs to increase the flow, since this is automatically implied by the system.

In some embodiments, the gas valve system may be adapted for switching from continuous flow to on demand flow, if the flow rate required is less than a predetermined level or the other way around.

In some other embodiments, the gas valve system may be adapted for indicating whether a patient can benefit from on-demand gas flow or whether continuous flow is required, e.g., depending on the type of breathing of the patient. Such a system may either automatically adjust or may provide an indication to the patient or the medical staff. In one example such a system may for example be adapted for facilitating the initiation of the therapy and giving relevant suggestions to a caregiver or patient after a short period of use. Such indications can be the selection of oxygen delivery mode such as continuous or on demand or the level of delivery. If breathing through the nose is for example detected and a regular breath rate such as between 8 and 20 or between 12 and 38 breaths per minute can be measured in the first 1 to 5 minutes, the on demand mode can be suggested or activated. If in these 1 to 5 minutes mouth or shallow breathing, no triggering, or a change in vital parameters such as breath rate, oxygen saturation and/or others (temperature, heart rate, blood pressure) are detected, continuous mode can be suggested or activated. In another example such a system may for example be adapted for following up the breathing type of a patient during therapy and giving relevant suggestions to a caregiver or patient after a longer period of use such as between 2 caregivers' parameter rounds, half a day or a few days. If breathing through the nose is sufficiently detected during this period and/or a regular breath rate such as between 8 and 20 or between 12 and 38 breaths per minute can be measured for a sufficient period of time, for example more than 8 hours a day, the on demand mode can be suggested or activated for that patient. If too long periods of mouth or shallow breathing, no triggering, or a change in vital parameters such as breath rate, oxygen saturation and/or others (temperature, heart rate, blood pressure) are detected, such as for example less than 8 hours per day, the continuous mode can be suggested or activated for a certain period of time such as 5 hours a day and then eventually rechanged in on demand mode after a period of time such as for the rest of the day.

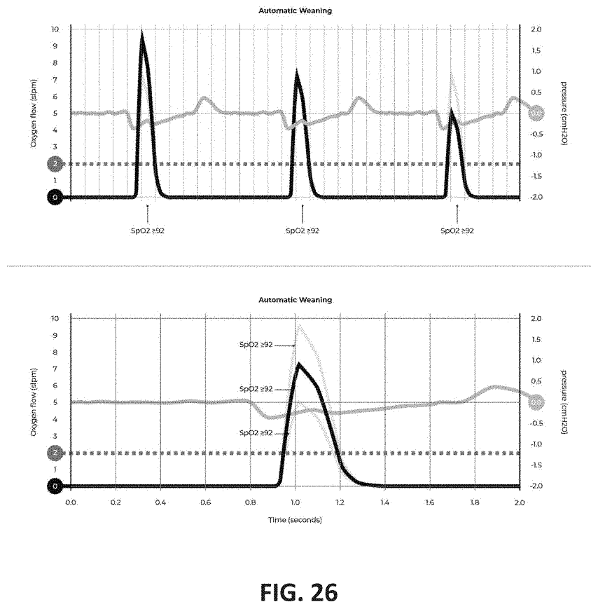

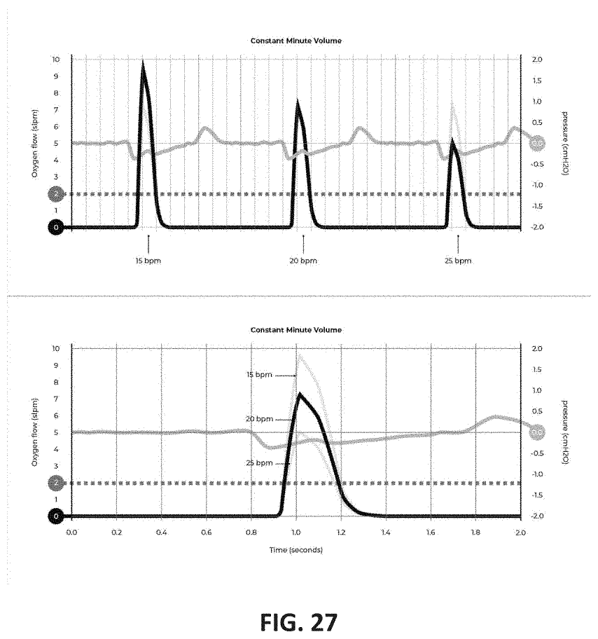

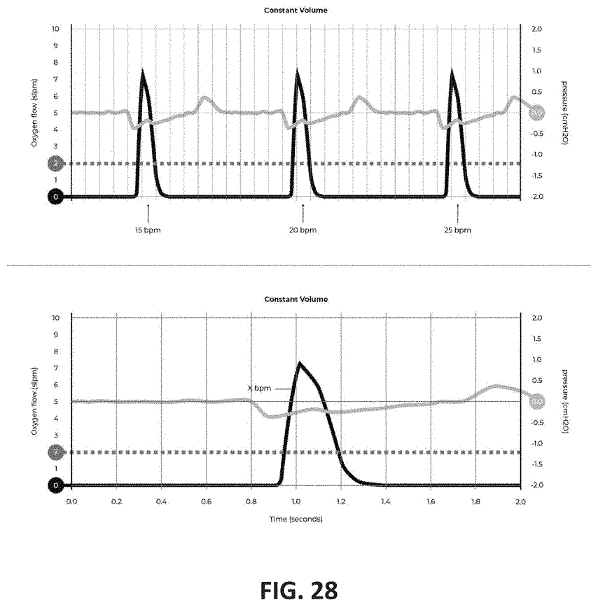

By way of illustration, embodiments of the present invention not being limited thereto, a number of possibilities for adjusting the release during the pulse in an on-demand operated gas valve system are shown. FIG. 25 illustrates the possibility of automatic increasing of the bolus volume during the release, when the user e.g., starts breathing faster. FIG. 26 illustrates the possibility of automatic decreasing, also referred to as automatic weaning, of the bolus volume during the release. FIG. 27 and FIG. 28 illustrate the possibility to obtain a constant minute volume that is delivered respectively a constant volume during the pulse release. It is to be noted that the above situations illustrated in FIG. 25 to FIG. 28 advantageously can be obtained with a single gas valve system, since the release during the pulse in the on-demand operated gas valve system can be adjusted withing the gas valve system.

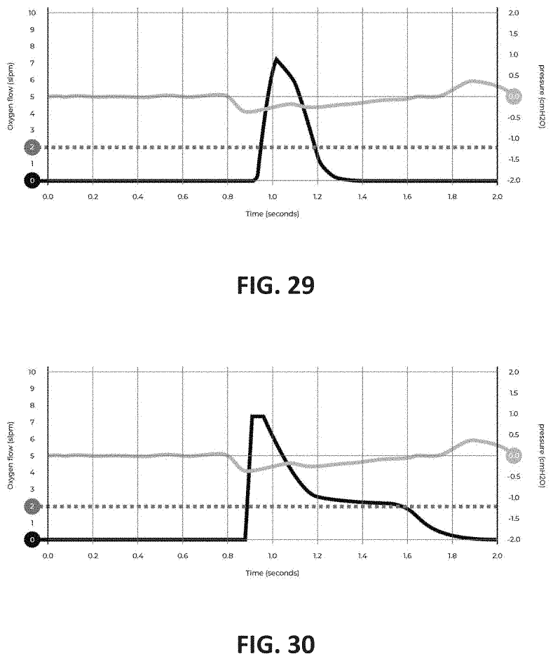

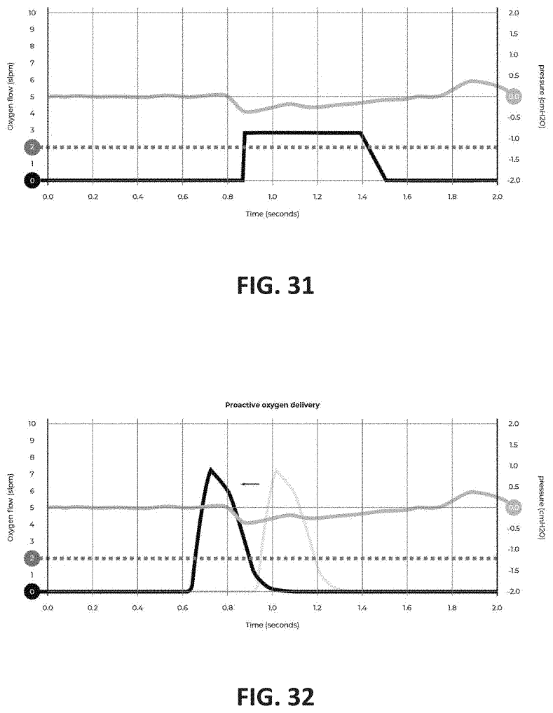

Furthermore, FIG. 29 to FIG. 31 illustrate other possibilities for adjusting the gas release during the pulse in an on-demand operated gas valve system, including adjusting the length of the release, the trigger time and or the bolus height.

FIG. 32 illustrates a further embodiment whereby, based on previous information of the breathing parameters of a user, the release can be controlled such that it is triggered shortly before the breathing in. The latter allows reduction of death volume during breathing in and results in a further optimised use of gas take up.

1. A gas valve system adapted for controlling the flow of a gas for a user, the gas valve system comprising:
a housing,
at least one sensor,
one or more electronic and/or mechanical components for controlling the gas flow, and
a manifold comprising one or more channels for guiding the gas between an input and an output of the gas valve system and to which the at least one sensor and the one or more electronic and/or mechanical components can be directly coupled.

2. The gas valve system according to claim 1, wherein the at least one sensor comprises at least a first sensor and at least a second sensor.

3. The gas valve system according to claim 1, wherein one or more of the channels in the manifold comprise one or more local channel diameter reductions.

4. The gas valve system according to claim 1, wherein the local channel diameter reduction is provided by local protrusions of the channel wall into the channel.

5. The gas valve system according to claim 4, wherein the channel comprises openings in the channel wall before and after the local channel diameter reduction, to which a flow sensor can be coupled for measuring the flow of the gas in a bypass channel.

6. The gas valve system according to claim 5, wherein the openings in the channel wall have an axial direction perpendicular to the overall longitudinal direction of the gas channel.

7. The gas valve system according to claim 6, wherein the openings in the channel wall have an axial direction non-perpendicular to the overall longitudinal direction of the gas channel.

8. The gas valve system according to claim 1, wherein the manifold is made of a single piece or a combination of pieces comprising the channels.

9. The gas valve system according to claim 1, wherein the manifold is made of any of a plastic or a metal.

10. The gas valve system according to claim 1, wherein the manifold comprises alignment features for aligning one of the one or more sensors and/or one or more electronic or mechanical components with respect to the channels in the manifold.

11. The gas valve system according to claim 1, the gas valve system comprising:
a local controller for controlling the flow and/or way of delivery of gas supplied to the user, and
a remotely positioned controller in communication with the local controller, the remote controller being configured for remotely obtaining information regarding the flow and/or the way of delivery of gas supplied to a user from the local controller and being configured for adjusting the local controller for adjusting the flow and/or way of delivery of the gas supplied to the user.

12. A manifold for use in a gas valve system, the manifold comprising one or more channels for guiding a gas between an input and an output of the gas valve system and to which at least one sensor and one or more electronic and/or mechanical components can be directly coupled.

13. The manifold according to claim 12, wherein the one or more channels in the manifold comprise one or more local channel diameter reductions.

14. The manifold according to claim 13, wherein the local channel diameter reduction is provided by local protrusions of the channel wall into the channel.

15. The manifold according to claim 14, wherein the channel comprises openings in the channel wall before and after the local channel diameter reduction, to which a flow sensor can be coupled for measuring the flow of the gas in a bypass channel.

16. The manifold according to claim 15, wherein the openings in the channel wall have an axial direction perpendicular to the overall longitudinal direction of the gas channel or wherein the openings in the channel wall have an axial direction non-perpendicular to the overall longitudinal direction of the gas channel.

17. A gas valve system adapted for controlling the flow of a gas for a user, the gas valve system comprising at least one sensor for measuring the output flow and or pressure of the gas supplied by the gas valve system, so as to determine the amount of gas delivered during a predetermined period.

18. The gas valve system according to claim 17, wherein the gas valve system comprises a memory for storing the amount of gas delivered during a predetermined period, or wherein the gas valve system comprises a user identification means and wherein the gas valve system is adapted for determining or storing the amount of gas delivered during a predetermined period for an identified user, for a given group of users, for a hospital or care institution department or for a hospital or care institution.

19. The gas valve system according to claim 17, wherein the gas valve system comprises a processor
for determining a degree of therapy loyalty of the user, the therapy loyalty expressing how good the user is applying a predescribed gas therapy, and/or
for identifying when a patient does not follow a predetermined gas therapy and/or
for identifying when a patient does not follow a predetermined gas therapy by means of a visual signal, an audio signal, a haptic signal or a gas pressure signal.

20. The gas valve system according to claim 17, wherein the gas valve system is furthermore configured for detecting one or more of a breathing type, an accurate positioning of a nasal canula, an accurate triggering.
